(19) United States
(12) Patent Application Publication
(10) Pub. No.: US 2026/0341530 A1
(43) Pub. Date: Oct. 29, 2026

(54) SIMULATION MODEL GENERATION DEVICE AND SIMULATION MODEL GENERATION METHOD

(71) Applicant: TERUMO KABUSHIKI KAISHA, Tokyo (JP)

(72) Inventor: Hiroya Sato, Ashigarakami-gun (JP)

(73) Assignee: TERUMO KABUSHIKI KAISHA, Tokyo (JP)

(21) Appl. No.: 19/707,572

(22) Filed: Jun. 15, 2026

Related U.S. Application Data
(63) Continuation of application No. PCT/JP2025/003346, filed on Feb. 3, 2025.

(30) Foreign Application Priority Data
Feb. 28, 2024 (JP) 2024-028742

(51) Int. Cl. G16H 50/50

(52) U.S. Cl. CPC G16H 50/50

(57) ABSTRACT

A simulation model generation device includes a processor configured to: acquire measurement data that is time-series data of a glucose level of a patient in a first period, acquire variation factor data, provisionally determine the variation factor data in a first period by allocating the variation factor data to each of a plurality of contribution events, set the variation factor data provisionally determined by the determination unit in a mathematical model, and generate a simulation model for estimating the glucose level of the patient.

### CROSS-REFERENCE TO RELATED APPLICATIONS

This is a bypass continuation of PCT Application No. PCT/JP2025/003346, filed on Feb. 3, 2025, which claims priority to Japanese Patent Application No. 2024-028742, filed on Feb. 28, 2024. The entire contents of these applications are hereby incorporated by reference herein.

### BACKGROUND

The present disclosure relates to a simulation model generation device and a simulation model generation method.

Japanese Patent No. 7290624 discloses a technique of estimating a blood glucose level of a patient on the basis of an amount of meal taken by the patient, an insulin dose of the patient, and the like.

### SUMMARY

It is desirable to satisfactorily estimate the blood glucose level of a patient.

An object of certain embodiments of the present invention is to solve the above-described problem.

(1) A first aspect of the present disclosure provides a simulation model generation device that includes: a first acquisition unit that acquires measurement data that is time-series data of a glucose level of a patient in a first period; a second acquisition unit that acquires variation factor data that is data pertaining to a variation factor inducing a variation in the glucose level of the patient in a second period included in the first period and shorter than the first period; a determination unit that provisionally determines the variation factor data in the first period by allocating the variation factor data acquired by the second acquisition unit to each of contribution events of the variation factor in a period that is included in the first period and not included in the second period; an optimization unit that sets the variation factor data provisionally determined by the determination unit in a mathematical model for obtaining estimation data that is time-series data of the glucose level of the patient, and performs optimization processing on a parameter included in the mathematical model so that the estimation data obtained by the mathematical model approximates to the measurement data acquired by the first acquisition unit to obtain an optimal solution of the parameter; and a generation unit that generates a simulation model for estimating the glucose level of the patient by setting the optimal solution of the parameter obtained by the optimization unit in the mathematical model.

In order to estimate the glucose level of the patient, it is necessary to set a parameter corresponding to the patient in advance in a predetermined mathematical model. Further, in order to increase the accuracy of calculating the glucose level, it is necessary to increase the accuracy of the parameter. In order to increase the accuracy of the parameter, it is desirable to specify the parameter using a sufficient amount of variation factor data (data pertaining to meal, data pertaining to insulin, etc.). However, the work of acquiring a sufficient amount of variation factor data and the work of manually inputting a sufficient amount of variation factor data into a device impose a burden on the patient or a user (doctor or the like).

In the configuration according to (1), the determination unit allocates the variation factor data in the second period shorter than the first period to each of contribution events of the variation factor in the period (third period) included in the first period and not included in the second period to provisionally determine the variation factor data in the first period. With this configuration, the patient does not need to acquire the variation factor data in the third period. In addition, the patient or the user (doctor or the like) does not need to input the variation factor data in the third period to the device. According to the configuration in (1), the burden on the patient or the user (doctor or the like) can be reduced. Therefore, according to the configuration in (1), the work of estimating the blood glucose level of the patient is simplified.

According to the configuration in (1), the optimal solution of the parameter included in the predetermined mathematical model can be obtained. That is, according to the configuration in (1), it is possible to generate a simulation model corresponding to the patient by setting the optimal solution of the parameter in the predetermined mathematical model. The user (doctor or the like) can satisfactorily estimate the blood glucose level of the patient by using a simulation model corresponding to the patient.

(2) In the simulation model generation device according to (1), the variation factor data acquired by the second acquisition unit may include meal amount data indicating an amount of meal ingested by the patient at each of a plurality of meal events during the second period, meal time data corresponding to each of the plurality of meal events during the second period, insulin dose data indicating a dose of insulin administered to the patient at each of a plurality of insulin administration events during the second period, and insulin administration time data corresponding to each of the plurality of insulin administration events during the second period.

(3) The simulation model generation device according to (1) or (2) may further include a distribution chart acquisition unit that generates the estimation data that is time-series data of the glucose level as data distribution information at each time.

(4) In the simulation model generation device according to (3), the second period may be 24 hours.

(5) A second aspect of the present disclosure provides a simulation model generation method including: a first acquisition step for acquiring measurement data that is time-series data of a glucose level of a patient in a first period; a second acquisition step for acquiring variation factor data that is data pertaining to a variation factor inducing a variation in the glucose level of the patient in a second period included in the first period and shorter than the first period; a determination step for provisionally determining the variation factor data in the first period by allocating the variation factor data acquired in the second acquisition step to each of contribution events of the variation factor in a period that is included in the first period and not included in the second period; an optimization step for setting the variation factor data provisionally determined in the determination step in a mathematical model for obtaining estimation data that is time-series data of the glucose level of the patient, and performing optimization processing on a parameter included in the mathematical model so that the estimation data obtained by the mathematical model approximates to the measurement data acquired in the first acquisition step to obtain an optimal solution of the parameter; and a generation step for generating a simulation model for estimating the glucose level of the patient by setting the optimal solution of the parameter obtained in the optimization step in the mathematical model.

In the configuration in (5), in the determination step, the variation factor data in the first period is provisionally determined by allocating the variation factor data in the second period shorter than the first period to each contribution event of the variation factor in the period (third period) included in the first period and not included in the second period. With this configuration, the patient does not need to acquire the variation factor data in the third period. In addition, the patient or the user (doctor or the like) does not need to input the variation factor data in the third period. According to the configuration in (5), the burden on the patient or the user (doctor or the like) can be reduced. Therefore, according to the configuration in (5), the work of estimating the blood glucose level of the patient is simplified.

According to the configuration in (5), the optimal solution of the parameter included in the predetermined mathematical model can be obtained. That is, according to the configuration in (5), it is possible to generate a simulation model corresponding to the patient by setting the optimal solution of the parameter in the predetermined mathematical model. The user (doctor or the like) can satisfactorily estimate the blood glucose level of the patient by using a simulation model corresponding to the patient.

(6) In the simulation model generation method according to (5), the variation factor data acquired in the second acquisition step may include meal amount data indicating an amount of meal ingested by the patient at each of a plurality of meal events during the second period, meal time data corresponding to each of the plurality of meal events during the second period, insulin dose data indicating a dose of insulin administered to the patient at each of a plurality of insulin administration events during the second period, and insulin administration time data corresponding to each of the plurality of insulin administration events during the second period.

(7) The simulation model generation method according to (5) or (6) may further include a distribution chart acquisition step for generating the estimation data that is time-series data of the glucose level as data distribution information at each time.

(8) In the simulation model generation method according to (7), the second period may be 24 hours.

(9) A third aspect of the present disclosure provides a non-transitory computer-readable medium comprising a program that, when executed, causes a computer to execute the simulation model generation method according to any one of (5) to (8).

According to certain embodiments of the present invention, the blood glucose level of a patient can be more satisfactorily estimated.

### BRIEF DESCRIPTION OF DRAWINGS

FIG. 1 is a schematic configuration diagram of a clinical decision-making support system;

FIG. 2 is a flowchart of simulation model generation processing;

FIG. 3 is a graph showing measurement data of a patient for seven days, and a meal amount and an insulin dose on the first day;

FIG. 4 is a graph showing measurement data of a patient for seven days, and meal amounts and insulin doses on the first to seven days; and

FIG. 5 is a graph displayed on a display unit.

### DETAILED DESCRIPTION

In the present specification, a subject whose glucose level (glucose concentration) is measured is referred to as a “patient.” An operator who operates a clinical decision-making support device is referred to as a “user.” For example, the user is any of a medical worker such as a doctor, a patient, and a person who cares for the patient. In the present specification, the term “glucose level” simply means the glucose level in the interstitial fluid under the skin of the patient. The glucose level in the interstitial fluid correlates with the blood glucose level. Therefore, the glucose level in the present specification may be treated as synonymous with the blood glucose level.

In order to estimate the glucose level of the patient, it is necessary to set a parameter corresponding to the patient in advance in a predetermined mathematical model. In order to increase the accuracy of calculating the glucose level to be estimated, it is necessary to increase the accuracy of estimating the parameter. In order to increase the accuracy of estimating the parameter, it is desirable to specify the parameter using a sufficient amount of data (for example, data pertaining to meal, data pertaining to insulin, etc.). However, the work of acquiring a sufficient amount of data and the work of inputting a sufficient amount of data into a device impose a burden on the patient or the user. The embodiment described below makes it possible to increase the accuracy of calculating the glucose level with the burden on the patient or the user being reduced.

### [1 Configuration of Clinical Decision-making Support System 10]

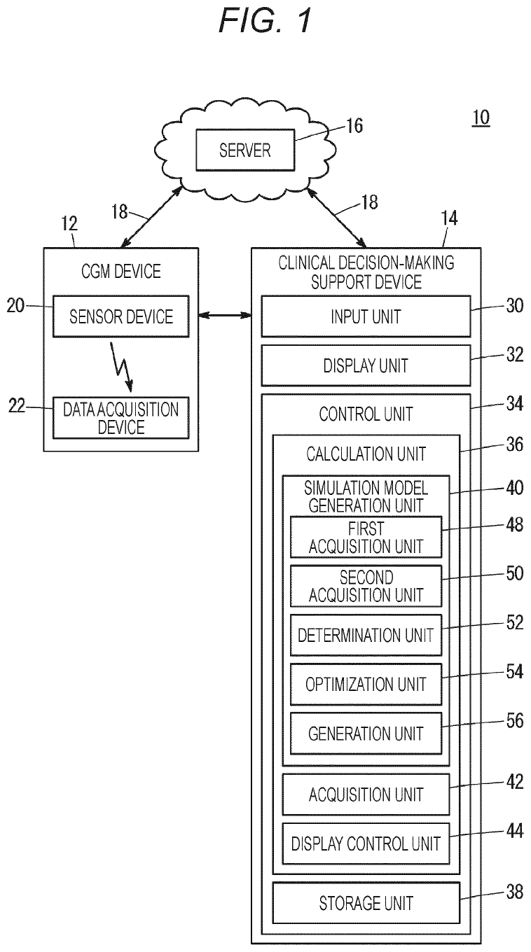

FIG. 1 is a schematic configuration diagram of a clinical decision-making support system 10. The clinical decision-making support system 10 includes a continuous glucose monitoring (CGM) device 12, a clinical decision-making support device 14, and a server 16. The CGM device 12 and the server 16 are communicably connected to each other via a communication line 18 such as the Internet. Similarly, the clinical decision-making support device 14 and the server 16 are communicably connected to each other via the communication line 18.

Note that, in the present specification, the clinical decision-making support system 10 including the server 16 will be described, but the server 16 is not a necessary configuration for the clinical decision-making support system 10. For example, in a case where the server 16 is not included in the clinical decision-making support system 10, the CGM device 12 and the clinical decision-making support device 14 are communicably connected to each other in a wireless or wired manner. For example, the CGM device 12 and the clinical decision-making support device 14 may be communicably connected to each other via the communication line 18, or may be communicably connected to each other via short-range wireless communication such as Bluetooth (registered trademark). Alternatively, the clinical decision-making support device 14 may read measurement data acquired by the CGM device 12 in a compatible file format.

### [1-1 CGM Device 12]

The CGM device 12 measures the glucose level in the interstitial fluid under the skin of the patient. The CGM device 12 includes a sensor device 20, which is an on-body device, and a data acquisition device 22. The sensor device 20 and the data acquisition device 22 are communicably connected to each other in a wired or wireless manner.

The sensor device 20 is placed on the body of the patient. The sensor device 20 includes a sensor and a transmitter (both not illustrated). The sensor continuously measures the glucose level in the interstitial fluid under the skin of the patient. The transmitter transmits a signal indicating the glucose level measured by the sensor to the data acquisition device 22.

The data acquisition device 22 is carried by the patient. The data acquisition device 22 may be, for example, a mobile terminal such as a smartphone. The data acquisition device 22 includes a receiver, a controller (a processor or the like), a monitor screen, a transmitter, a memory, and an input unit (all not illustrated). The receiver receives a signal indicating a glucose level transmitted from the sensor device 20. The controller displays the glucose level of the patient on the monitor screen. The transmitter transmits a signal indicating the glucose level to the server 16 via the communication line 18. The memory stores time-series glucose levels. The patient may input variation factor data to the data acquisition device 22 using the input unit.

### [1-2 Clinical Decision-making Support Device 14]

The clinical decision-making support device 14 is an information processing device having a function of simulating a blood glucose trend of the patient. In addition, the clinical decision-making support device 14 has a function of generating a simulation model for simulating a blood glucose trend of the patient. That is, the clinical decision-making support device 14 also functions as a simulation model generation device. The clinical decision-making support device 14 simulates a blood glucose trend of the patient after generating a simulation model corresponding to the patient. The clinical decision-making support device (simulation model generation device) 14 generates a simulation model corresponding to the patient on the basis of “measurement data” and “variation factor data.” Furthermore, the clinical decision-making support device 14 obtains “estimation data” by setting “variation factor data” of the patient in the simulation model. Prior to describing the configuration of the clinical decision-making support device 14, the name of each data is defined.

The measurement data is time-series data of the glucose level of the patient in a first period (for example, three days, one week, one month, or the like). The measurement data is the concentration of glucose in the interstitial fluid of the patient measured continuously over a prescribed period of time. The measurement data is usually acquired once every one to five minutes. In the present embodiment, the measurement data is time-series data of the glucose level measured by the CGM device 12, and is stored in the server 16.

The variation factor data is data related to a variation factor in a second period (for example, 24 hours) included in the first period and shorter than the first period. The variation factor is an action (disturbance) of the patient that has induced a variation in the glucose level of the patient, and is, for example, meal intake and administration of insulin. That is, the variation factor data includes meal data regarding meal intake and insulin data regarding insulin administration. The meal data includes meal amount data and meal time data corresponding to the meal amount data. The insulin data includes insulin dose data and insulin administration time data corresponding to the insulin dose data.

The meal amount data is data indicating the amount of carbohydrate or the amount of sugar ingested by the patient in each of a plurality of meal events (breakfast, lunch, dinner, snack, etc.) in the second period (24 hours). The meal time data is data indicating a time at which each of the plurality of meal events occurred in the second period. The insulin dose data is data indicating the dose of insulin administered to the patient in each of a plurality of insulin administration events (before meal, during meal, before bedtime, etc.) during the second period. The insulin administration time data is data indicating the time at which each of the plurality of insulin administration events occurred during the second period.

The patient can store the variation factor data in the server 16 via, for example, a communication terminal (the CGM device 12, a personal computer (not illustrated), or the like) owned by the patient.

The estimation data is time-series data of the glucose level of the patient obtained by the simulation model. The estimation data is obtained by setting variation factor data of the patient in an arbitrary period in a simulation model corresponding to the patient.

The clinical decision-making support device 14 acquires time-series data and variation factor data from the server 16 via the communication line 18. In the present specification, an embodiment in which the variation factor data is acquired from the server 16 will be described, but the variation factor data may be input by a user (doctor or the like) operating the input unit 30. For example, the patient may report the variation factor data to the user. In this case, the user operates the input unit 30 to input the variation factor data reported by the patient to a control unit 34 (storage unit 38).

The clinical decision-making support device 14 may be, for example, a terminal such as a personal computer or a tablet. The clinical decision-making support device 14 includes the input unit 30, a display unit 32, and the control unit 34. The control unit 34 further includes a calculation unit 36 and the storage unit 38.

The input unit 30 is a user machine interface operable by the user. The input unit 30 may include a mouse, a touch panel, a keyboard, a voice input device, and the like. The input unit 30 transmits a signal corresponding to an input operation by the user to the control unit 34. The display unit 32 displays a video corresponding to a video signal transmitted from the control unit 34.

The calculation unit 36 of the control unit 34 can be configured by, for example, a processor such as a central processing unit (CPU) or a graphics processing unit (GPU). That is, the calculation unit 36 can be configured by processing circuitry. At least a part of the calculation unit 36 may be implemented by an integrated circuit such as an application specific integrated circuit (ASIC) or a field-programmable gate array (FPGA). At least a part of the calculation unit 36 may be implemented by an electronic circuit including a discrete device.

The calculation unit 36 includes a simulation model generation unit 40, an acquisition unit (distribution chart acquisition unit) 42, and a display control unit 44. The simulation model generation unit 40, the acquisition unit 42, and the display control unit 44 can be implemented by executing a program stored in the storage unit 38 by the calculation unit 36.

The simulation model generation unit 40 specifies a parameter corresponding to the patient by performing optimization processing of optimizing a parameter included in a predetermined mathematical model for estimating a glucose level. As a result, the simulation model generation unit 40 generates a mathematical model including the parameter corresponding to the patient. A predetermined mathematical model including the parameter corresponding to the patient is referred to as a simulation model corresponding to the patient. A specific example of the predetermined mathematical model will be described later.

The simulation model generation unit 40 includes a first acquisition unit 48, a second acquisition unit 50, a determination unit 52, an optimization unit 54, and a generation unit 56. The first acquisition unit 48 acquires the measurement data measured by the CGM device 12 during the first period (for example, one week, one month, or the like) from the server 16 in response to the operation on the input unit 30 by the user. The second acquisition unit 50 acquires the variation factor data in the second period (for example, 24 hours or the like) from the server 16 in response to the operation on the input unit 30 or the data acquisition device 22 of the CGM device 12 by the user. Alternatively, the second acquisition unit 50 reads the variation factor data directly input to the input unit 30.

The determination unit 52 allocates the variation factor data acquired by the second acquisition unit 50 to each of contribution events (for example, a meal event, or an insulin administration event) of the variation factor in a third period included in the first period and not included in the second period. Accordingly, the determination unit 52 provisionally determines the variation factor data in the third period.

The optimization unit 54 performs optimization processing for obtaining optimal solutions of the variation factor in the first period (second period+third period) and the parameter included in the predetermined mathematical model. The optimization unit 54 optimizes the variation factor data in the first period provisionally determined by the determination unit 52 with respect to the measurement data in the first period. Furthermore, the optimization unit 54 optimizes the variation factor data and the parameter so that the measurement data is approximated with a predetermined mathematical model. By performing these processes, the optimization unit 54 obtains an optimal solution of the parameter included in the predetermined mathematical model. The predetermined mathematical model will be described later.

The generation unit 56 generates a simulation model for estimating the glucose level of the patient by setting the optimal solution of the parameter obtained by the optimization unit 54 in the predetermined mathematical model. The simulation model generated here is a simulation model corresponding to the patient.

The acquisition unit 42 sets the variation factor data input via the input unit 30 or the variation factor data stored in the storage unit 38 in the simulation model corresponding to the patient generated by the simulation model generation unit 40, thereby acquiring estimation data.

The display control unit 44 performs display control for displaying various types of information on the display unit 32. For example, the display control unit 44 performs display control for displaying the estimation data acquired by the acquisition unit 42 on the display unit 32. The display control unit 44 transmits a video signal for displaying various types of information to the display unit 32.

The storage unit 38 is a computer-readable storage medium. The storage unit 38 includes a volatile memory (not illustrated) and a nonvolatile memory (not illustrated). The volatile memory is, for example, a random access memory (RAM) or the like. The nonvolatile memory is, for example, a read only memory (ROM), a flash memory, or the like. The volatile memory stores data and the like. The nonvolatile memory stores programs, tables, maps, and the like. At least a part of the storage unit 38 may be provided in the above-described processor, integrated circuit, or the like.

The storage unit 38 is a storage medium that stores a program for implementing each unit in the calculation unit 36. The storage unit 38 is also a storage medium that stores a program for executing simulation model generation processing and clinical decision-making support processing described below.

### [1-3 Server 16]

The server 16 can be provided as a physical server or a cloud server. The server 16 includes a processor and a memory (both not illustrated).

The server 16 acquires measurement data of the patient from the data acquisition device 22 of the CGM device 12 via the communication line 18. Furthermore, the server 16 acquires variation factor data of the patient from a communication terminal (data acquisition device 22, personal computer (not illustrated), or the like) owned by the patient via the communication line 18. The measurement data and the variation factor data are stored in the memory. The server 16 transmits a signal indicating the measurement data and the variation factor data to the clinical decision-making support device 14 via the communication line 18.

### [2 Mathematical Model]

As described above, the optimization unit 54 of the simulation model generation unit 40 performs optimization processing for obtaining an optimal solution of a parameter included in a predetermined mathematical model. As the mathematical model, for example, Hovorka model disclosed in Japanese Patent No. 7290624 or the like or an equation disclosed in the specification of U.S. Pat. No. 6,923,763 or the like can be used.

For example, the Hovorka model is the following simultaneous ordinary differential equations with time t as a variable. In the following simultaneous ordinary differential equations, the function i(t) is the dose of insulin. The function cho(t) is the amount of meal (amount of carbohydrate). The function G(t) is a glucose level.

$$\frac{d {S}_{1}}{d t} = i ( t ) - {k}_{a} \cdot {S}_{1} ( t ) \qquad [ Mathematical Expression 1 ]$$

$$\frac{d {S}_{2}}{d t} = {k}_{a} \cdot {S}_{1} ( t ) - {k}_{a} \cdot {S}_{2} ( t )$$

$$\frac{d I}{d t} = \frac{{k}_{a} \cdot {S}_{2} ( t )}{{V}_{I}} - {k}_{e} \cdot I ( t )$$

$$\frac{d {D}_{1}}{d t} = c h o ( t ) - \frac{{D}_{1} ( t )}{{t}_{max}}$$

$$\frac{d {D}_{2}}{d t} = \frac{{D}_{1} ( t )}{{t}_{max}} - \frac{{D}_{2} ( t )}{{t}_{max}}$$

$${U}_{G} = \frac{{D}_{2} ( t )}{{t}_{max}}$$

$$\frac{d {Q}_{1}}{d t} = - [ \frac{{F}_{01}^{C}}{{V}_{G} \cdot G ( t )} + {x}_{1} ( t ) ] \cdot {Q}_{1} ( t ) + {k}_{12} {Q}_{2} ( t ) - {F}_{R} + E G {P}_{0} \cdot [ 1 - {x}_{3} ( t ) ] + {U}_{G} ( t )$$

$$\frac{d {Q}_{2}}{d t} = {x}_{1} ( t ) \cdot {Q}_{i} ( t ) - [ {k}_{12} + {x}_{2} ( t ) ] \cdot {Q}_{2} ( t )$$

$$\frac{d {x}_{1}}{d t} = - {k}_{a 1} \cdot {x}_{1} ( t ) + {k}_{b 1} \cdot I ( t )$$

$$\frac{d {x}_{2}}{d t} = - {k}_{a 2} \cdot {x}_{2} ( t ) + {k}_{b 2} \cdot I ( t )$$

$$\frac{d {x}_{3}}{d t} = - {k}_{a 3} \cdot {x}_{3} ( t ) + {k}_{b 3} \cdot I ( t )$$

$$G ( t ) = \frac{{Q}_{1} ( t )}{{V}_{G}}$$

In the simultaneous ordinary differential equations, F₀₁^{C} is expressed by the following expression.

$${F}_{01}^{C} = \frac{{F}_{01} \cdot G ( t )}{0.85 ( G ( t ) + 1.0 )} \qquad [ Mathematical Expression 2 ]$$

In the simultaneous ordinary differential equations, F_{R} is 0.003 (G−9)×V_{G} when G>9, and is 0 when G>9 is not established.

The simultaneous ordinary differential equations include 15 parameters: V_{G}, F₀₁, k₁₂, F_{R}, EGP₀, k_{b1}, kₐ₁, k_{b2}, kₐ₂, k_{b3}, kₐ₃, kₐ, V_{I}, kₑ, and tₘₐₓ. The parameters are as follows.
V_{G}: Volume of the distribution of glucose (unit: liter)
F₀₁: Non-insulin-dependent glucose transfer rate (unit: mmol/min)
k₁₂: Constant of transfer rate of glucose from tissue to blood (unit: min⁻¹)
kₐ₁, kₐ₂, kₐ₃: Insulin inactivation rate constant (unit: min⁻¹)
F_{R}: Constant of urinary excretion of glucose (unit: mmol/min)
EGP₀: Endogenous glucose production rate per unit time (unit: min⁻¹)
k_{b1}, k_{b2}, k_{b3}: Insulin activation rate constant (unit: min⁻¹)
kₐ: Rate of absorption of insulin injected subcutaneously (unit: min⁻¹)
V_{I}: Volume of distribution of insulin (unit: liter)
kₑ: Plasma elimination rate of insulin (unit: min⁻¹)
tₘₐₓ: Time to reach the peak of absorption of glucose ingested by the patient (unit: min)

The storage unit 38 stores a predetermined mathematical model. For example, the storage unit 38 stores the simultaneous ordinary differential equations described above.

### [3 Simulation Model Generation Processing]

Before the simulation model generation unit 40 of the clinical decision-making support device 14 generates a simulation model corresponding to the patient, the CGM device 12 acquires measurement data during the first period. The server 16 acquires the measurement data during the first period from the CGM device 12. Furthermore, before the simulation model generation processing, the patient inputs the variation factor data in the second period included in the first period and shorter than the first period to the communication terminal (for example, CGM device 12) owned by the patient. The server 16 acquires the variation factor data during the second period from the communication terminal owned by the patient. The server 16 stores the measurement data and the variation factor data acquired from the communication terminal owned by the patient. Alternatively, the user can also directly manually input the variation factor data during the second period to the input unit 30 of the clinical decision-making support device 14. In this case, it is only sufficient that variation factor data for one day is input as the variation factor data during the second period. Note that the variation factor data may be input by the patient or may be input by another user such as a medical worker.

Note that the patient may input information indicating specific meal contents (for example, the name of the meal menu, the actual intake amount) to the communication terminal (for example, CGM device 12) instead of the meal amount data in the variation factor data. In order to respond to such a case, the memory of the communication terminal or the memory of the server 16 can store in advance a conversion table for converting information indicating meal contents into meal amount data. Alternatively, the communication terminal can acquire information for converting the information indicating the meal contents into meal amount data from anywhere other than the server 16. As a result, the communication terminal or the server 16 can acquire meal amount data based on the conversion table when the information indicating meal contents is input.

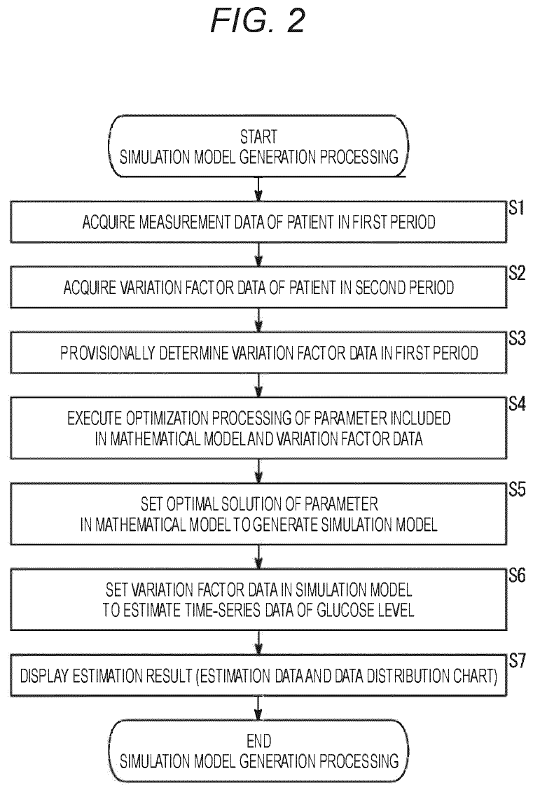

FIG. 2 is a flowchart of simulation model generation processing. The simulation model generation processing is executed by the clinical decision-making support device (simulation model generation device) 14. The user (doctor or the like) performs a predetermined operation on the input unit 30. As a result, the input unit 30 instructs the calculation unit 36 of the control unit 34 to execute the simulation model generation processing. The calculation unit 36 executes the simulation model generation processing illustrated in FIG. 2 according to an instruction signal output from the input unit 30.

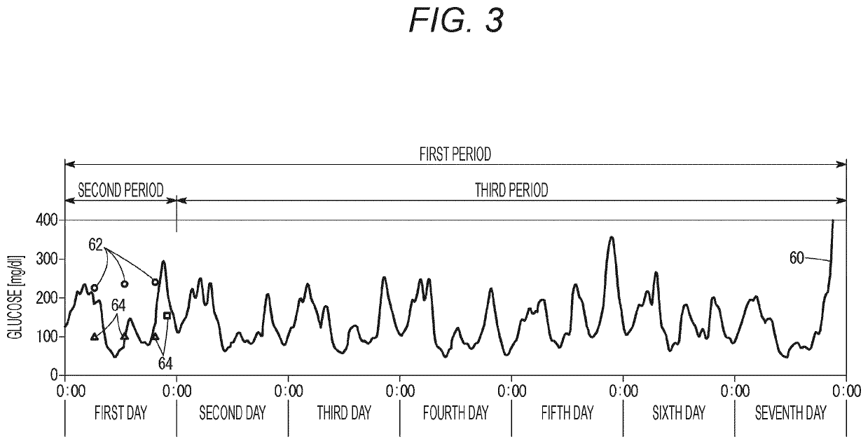

In step S1, the first acquisition unit 48 acquires the measurement data of the patient in the first period from the server 16 via the communication line 18. Alternatively, the first acquisition unit 48 may directly acquire the measurement data of the patient from the CGM device 12 (upon acquiring the measurement data, the data acquisition device 22 may be used). For example, a curve 60 in FIG. 3 shows measurement data for seven days of the patient.

In step S2, the second acquisition unit 50 acquires the variation factor data of the patient in the second period from the server 16 via the communication line 18. The second acquisition unit 50 may acquire the variation factor data input from the input unit 30. Three points 62 in FIG. 3 indicate meal data (meal amount data and meal time data) on the first day (24 hours) of the first period (for example, seven days). Four points 64 in FIG. 3 indicate insulin data (insulin dose data and insulin administration time data) on the first day of the first period. As described above, the second period is shorter than the first period. As the variation factor data, information stored in the server 16 may be used. As the variation factor data, the user may directly input the representative value on the first day of the first period using the input unit 30. For example, in a case where the variation factor data is meal data, representative values of the amount of carbohydrate (sugar intake) recognized as being ingested by the patient and the meal time data are input on the basis of the information self-reported by the patient. Alternatively, in a case where the variation factor data is insulin data related to the administration of insulin, the insulin dose data and the insulin administration time data are directly input based on the proposed prescription. Note that the variation factor data is not limited to the meal data and the insulin data, and information such as the type of exercise, the amount of exercise, and fever may be further added.

In step S3, the determination unit 52 provisionally determines the variation factor data in the first period. As described above, the period included in the first period and not included in the second period is referred to as a third period. For example, meal data (meal amount data and meal time data) of each meal event on the first day (second period) acquired in step S2 is provisionally input as an initial value of meal data of each meal event on each of the second to the seventh days (third period) by the determination unit 52. That is, the determination unit 52 also allocates the meal data of the breakfast event on the first day to the breakfast event on each of the second to the seventh days. In addition, the determination unit 52 also allocates the meal data of the lunch event on the first day to the lunch event on each of the second to the seventh days. In addition, the determination unit 52 also allocates the meal data of the dinner event on the first day to the dinner event on each of the second to the seventh days. Regarding snacking other than breakfast, lunch, and dinner, the determination unit 52 also allocates the meal data of the snacking event on the first day to the snacking event on each of the second to the seventh days.

Similarly, the determination unit 52 also uses the insulin data (insulin dose data and insulin administration time data) of the insulin administration event on the first day (second period) acquired in step S2 as the insulin data of each insulin administration event on each of the second to the seventh days (third period). That is, the determination unit 52 also allocates the insulin data of the insulin administration event at breakfast on the first day to the insulin administration event at breakfast on each of the second to the seventh days. In addition, the determination unit 52 also allocates the insulin data of the insulin administration event at lunch on the first day to the insulin administration event at lunch on each of the second to the seventh days. In addition, the determination unit 52 also allocates the insulin data of the insulin administration event at dinner on the first day to the insulin administration event at dinner on each of the second to the seventh days. In addition, the determination unit 52 also allocates the insulin data of the insulin administration event before bedtime on the first day to the insulin administration event before bedtime on each of the second to the seventh days.

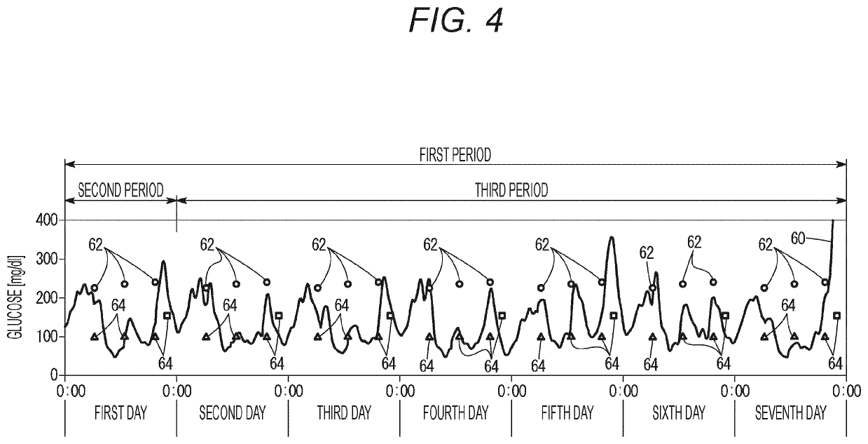

By the process of step S3 described above, the variation factor data of seven days (first period) is provisionally determined as illustrated in FIG. 4.

In step S4, the optimization unit 54 executes processing (optimization processing) of optimizing parameters included in the predetermined mathematical model (for example, the simultaneous ordinary differential equations) and the variation factor data. With this process, the optimization unit 54 acquires a parameter corresponding to the measurement data of the patient acquired by the first acquisition unit 48 in step S1. This parameter can be treated as a parameter corresponding to the patient. At the end of step S3, the parameter included in the predetermined mathematical model is unknown. If the parameter corresponding to the patient is set to the predetermined mathematical model, time-series estimation data obtained by setting the variation factor data in the first period in the predetermined mathematical model should approximate to the measurement data acquired in step S1. In view of this, the optimization unit 54 sets the variation factor data in the first period acquired in step S3 in the predetermined mathematical model including an unknown parameter. Then, the optimization unit 54 performs optimization processing of optimizing the parameter in the mathematical model such that the time-series estimation data obtained by the predetermined mathematical model approximates to the measurement data acquired in step S1. The optimization unit 54 performs optimization processing using at least one of predetermined optimization methods (for example, a steepest descent method, a quasi-Newton method, a Newton method, a Markov chain Monte Carlo method, Bayesian optimization, and the like). Thus, the optimization unit 54 obtains an optimal solution of the parameter included in the predetermined mathematical model. Furthermore, the optimization unit 54 performs optimization processing on each data included in the variation factor data using at least one of predetermined optimization methods in order to obtain an optimal solution of the variation factor data provisionally determined in step S3. Steps S3 and S4 may be executed a plurality of times. As a result, the optimization unit 54 finely adjusts the provisionally determined variation factor data.

In step S5, the generation unit 56 sets the optimal solution of the parameter obtained by the optimization unit 54 in a predetermined mathematical model. With this process, the generation unit 56 generates a simulation model for estimating the glucose level of the patient. The generation unit 56 stores the generated simulation model and the finely adjusted variation factor data obtained by the optimization unit 54 in the storage unit 38.

In step S6, the acquisition unit 42 estimates the time-series data of the glucose level by setting the variation factor data in the simulation model. Specifically, the acquisition unit 42 sets the variation factor data determined in step S4 (the variation factor data stored in the storage unit 38) in the simulation model generated in step S5 to acquire the estimation data. The estimation data may be generated over the same period as the first period. Note that the acquisition unit 42 can acquire data obtained by analyzing the estimation data by a statistical method in any display format. In this case, for example, the variation or imbalance in the 24-hour blood glucose fluctuations can be expressed using a known distribution expression method (for example, box-and-whisker plot, percentile curve, and the like at any time). When a box-and-whisker plot is used, outliers may be displayed together. In the following, an analysis result indicating the 24-hour blood glucose level distribution obtained by the analysis of the estimation data is referred to as a data distribution chart. The acquisition unit 42 may convert the estimation data into blood glucose level data.

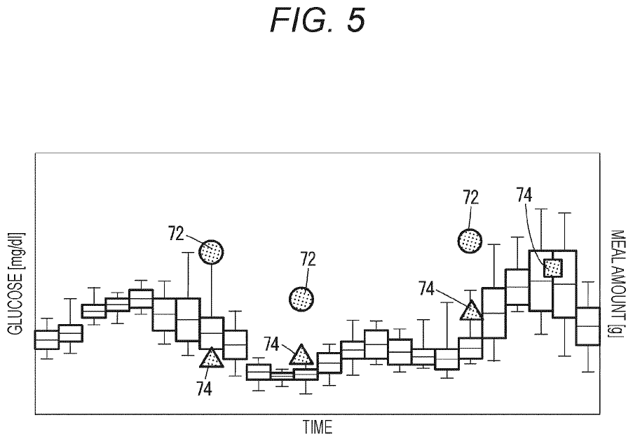

In step S7, the display control unit 44 causes the display unit 32 to display the estimation result in step S6. Specifically, the display control unit 44 transmits a video signal for displaying the data distribution chart acquired in step S6 and the variation factor data determined in step S4 to the display unit 32. Thus, the display unit 32 displays the data distribution chart and the variation factor data. For example, the display unit 32 displays the graph illustrated in FIG. 5. This graph shows information corresponding to the estimation data and the variation factor data. The graph represents 24-hour blood glucose fluctuations.

FIG. 5 illustrates a graph displayed by the display unit 32. The data distribution chart illustrated in FIG. 5 represents data distribution in each time zone. Each of three points 72 illustrated in FIG. 5 indicates meal data (meal amount data and meal time data) stored in the storage unit 38. Each of four points 74 in FIG. 5 indicates insulin data (insulin dose data and insulin administration time data) stored in the storage unit 38. As the initial value of the meal data and the initial value of the insulin data, variation factor data reported by the patient or a value after the optimization processing may be used.

As shown in FIG. 5, when the insulin data and the meal data including time information are displayed in the display region of the data distribution chart representing the 24-hour blood glucose fluctuations, it is easy to visually grasp the relationship between the daily treatment and the blood glucose trend. The points 72 and 74 may not be displayed in the frame of the graph. In addition, display fields indicating meal amount data, meal time data, insulin dose data, and insulin administration time data may be separately set in the vicinity of the graph. In the above description, three pieces of meal data and four pieces of insulin data are used per day, but one piece of meal data or two or more pieces of meal data may be used. The meal data may be a number corresponding to the total of the number of meals and the number of snacks per day. The insulin data may be any number of times of administration.

Note that, in the simulation model generation processing described above, steps S6 and S7 are processing for the user to confirm estimation data obtained when the optimized variation factor data is set in the simulation model.

As described above, in the clinical decision-making support device 14, the determination unit 52 allocates the variation factor data in the second period shorter than the first period to each contribution event of the variation factor in the third period included in the first period and not included in the second period to provisionally determine the variation factor data in the first period. Therefore, the patient does not need to separately acquire the variation factor data in the third period. In addition, the patient or the user (doctor or the like) does not need to input the variation factor data in the third period to the device. According to the clinical decision-making support device 14, it is possible to reduce the trouble of the user recording the meal amount and the meal time over a plurality of days and the trouble of the user inputting the meal amount and the meal time to a recording device such as the data acquisition device 22. Therefore, the clinical decision-making support device 14 simplifies the work for estimating the blood glucose level of the patient.

According to the clinical decision-making support device 14, it is possible to obtain an optimal solution of a parameter included in a predetermined mathematical model. That is, according to the clinical decision-making support device 14, it is possible to generate a simulation model corresponding to the patient by setting the optimal solution of the parameter in the predetermined mathematical model. The user can satisfactorily estimate the blood glucose level of the patient by using a simulation model corresponding to the patient. More specifically, if the user arbitrarily inputs the variation factor data on the first day of the first period using the input unit 30 as the variation factor data in step S2, it is possible to accurately obtain time-series estimation data based on the input value each time. Furthermore, according to the clinical decision-making support device 14, a data distribution chart based on the estimation data can be displayed. As a result, it is possible to simulate the meal amount, the meal time, the insulin dose, and the insulin administration time while showing the patient the data distribution chart estimated based on the specific meal data and insulin data, and thus it is easy for the patient to work on the treatment with conviction. In this manner, a simulation model with high accuracy can be acquired on the basis of the actual measurement data of each patient.

The clinical decision-making support device 14 can simulate the blood glucose trend of the patient using the generated simulation model.

While the present disclosure has been described in detail, the present disclosure is not limited to the individual embodiments described above. Various additions, substitutions, modifications, partial deletions, and the like may be made to these embodiments without departing from the gist of the present disclosure or without departing from the spirit of the present disclosure derived from the contents described in the claims and equivalents thereof. Further, the embodiments can be implemented in combination. For example, the order of operations and the order of processes in the above embodiments are provided as an example, and they are not limited to those described in the embodiments. The same applies to the case where numerical values or expressions are used in the description of the above embodiments.

What is claimed is:

1. A simulation model generation device comprising:
a processor configured to:
acquire measurement data that is time-series data of a glucose level of a patient in a first period,
acquire variation factor data that is data pertaining to a variation factor inducing a variation in the glucose level of the patient in a second period included in the first period and shorter than the first period,
provisionally determine the variation factor data in the first period by allocating the variation factor data acquired by the second acquisition unit to each of a plurality of contribution events that contribute to the variation factor and occur in a third period that is included in the first period and not included in the second period,
set the variation factor data provisionally determined by the determination unit in a mathematical model for obtaining estimation data that is time-series data of the glucose level of the patient, and performs optimization processing on a parameter included in the mathematical model so that the estimation data obtained by the mathematical model approximates to the measurement data acquired by the first acquisition unit to obtain an optimal solution of the parameter, and
generate a simulation model for estimating the glucose level of the patient by setting the optimal solution of the parameter obtained by the optimization unit in the mathematical model.

2. The simulation model generation device according to claim 1, wherein:
the variation factor data acquired by the second acquisition unit includes:
meal amount data indicating an amount of meal ingested by the patient at each of a plurality of meal events during the second period,
meal time data corresponding to each of the plurality of meal events during the second period,
insulin dose data indicating a dose of insulin administered to the patient at each of a plurality of insulin administration events during the second period, and
insulin administration time data corresponding to each of the plurality of insulin administration events during the second period.

3. The simulation model generation device according to claim 1, wherein:
the processor is further configured to generate the estimation data that is time-series data of the glucose level as data distribution information at each time.

4. The simulation model generation device according to claim 2, further comprising:
the processor is further configured to generate the estimation data that is time-series data of the glucose level as data distribution information at each time.

5. The simulation model generation device according to claim 3, wherein
the second period is 24 hours.

6. The simulation model generation device according to claim 4, wherein
the second period is 24 hours.

7. A simulation model generation method comprising:
acquiring measurement data that is time-series data of a glucose level of a patient in a first period;
acquiring variation factor data that is data pertaining to a variation factor inducing a variation in the glucose level of the patient in a second period included in the first period and shorter than the first period;
provisionally determining the variation factor data in the first period by allocating the acquired variation factor data to each of a plurality of contribution events that contribute to the variation factor and occur in a third period that is included in the first period and not included in the second period;
setting the provisionally determined variation factor data in a mathematical model for obtaining estimation data that is time-series data of the glucose level of the patient, and performing optimization processing on a parameter included in the mathematical model so that the estimation data obtained by the mathematical model approximates to the acquired measurement data to obtain an optimal solution of the parameter; and
a generating a simulation model for estimating the glucose level of the patient by setting the optimal solution of the parameter obtained in the optimization step in the mathematical model.

8. The simulation model generation method according to claim 7, wherein
the variation factor data includes:
meal amount data indicating an amount of meal ingested by the patient at each of a plurality of meal events during the second period,
meal time data corresponding to each of the plurality of meal events during the second period,
insulin dose data indicating a dose of insulin administered to the patient at each of a plurality of insulin administration events during the second period, and
insulin administration time data corresponding to each of the plurality of insulin administration events during the second period.

9. The simulation model generation method according to claim 7, further comprising:
generating the estimation data that is time-series data of the glucose level as data distribution information at each time.

10. The simulation model generation method according to claim 8, further comprising:
generating the estimation data that is time-series data of the glucose level as data distribution information at each time.

11. The simulation model generation method according to claim 9, wherein
the second period is 24 hours.

12. The simulation model generation method according to claim 10, wherein
the second period is 24 hours.

13. A non-transitory computer-readable medium comprising a program that, when executed, causes a computer to execute a method comprising:
acquiring measurement data that is time-series data of a glucose level of a patient in a first period;
acquiring variation factor data that is data pertaining to a variation factor inducing a variation in the glucose level of the patient in a second period included in the first period and shorter than the first period;
provisionally determining the variation factor data in the first period by allocating the acquired variation factor data to each of a plurality of contribution events that contribute to the variation factor and occur in a third period that is included in the first period and not included in the second period;
setting the provisionally determined variation factor data in a mathematical model for obtaining estimation data that is time-series data of the glucose level of the patient, and performing optimization processing on a parameter included in the mathematical model so that the estimation data obtained by the mathematical model approximates to the acquired measurement data to obtain an optimal solution of the parameter; and
a generating a simulation model for estimating the glucose level of the patient by setting the optimal solution of the parameter obtained in the optimization step in the mathematical model.

14. The non-transitory computer-readable medium according to claim 13, wherein
the variation factor data includes:
meal amount data indicating an amount of meal ingested by the patient at each of a plurality of meal events during the second period,
meal time data corresponding to each of the plurality of meal events during the second period,
insulin dose data indicating a dose of insulin administered to the patient at each of a plurality of insulin administration events during the second period, and
insulin administration time data corresponding to each of the plurality of insulin administration events during the second period.

15. The non-transitory computer-readable medium according to claim 13, further comprising:
generating the estimation data that is time-series data of the glucose level as data distribution information at each time.

16. The non-transitory computer-readable medium according to claim 14, further comprising:
generating the estimation data that is time-series data of the glucose level as data distribution information at each time.

17. The non-transitory computer-readable medium according to claim 15, wherein
the second period is 24 hours.

18. The non-transitory computer-readable medium according to claim 16, wherein
the second period is 24 hours.
